(19) United States
(12) Patent Application Publication
(10) Pub. No.: US 2025/0011690 A1
(43) Pub. Date: Jan. 9, 2025

(54) MICROCAPSULES

(71) Applicant: IBERCHEM S.A.U., Alcantarilla Murcia (ES)

(72) Inventors: Michael John Robert WHITE, Alcantarilla Murcia (ES); Sandra CERMEÑO OLMOS, Alcantarilla Murcia (ES)

(73) Assignee: IBERCHEM S.A.U., Alcantarilla Murcia (ES)

(21) Appl. No.: 18/697,923

(22) PCT Filed: Sep. 23, 2022
(86) PCT No.: PCT/EP2022/076527

§ 371 Date: Apr. 2, 2024

(30) Foreign Application Priority Data
Oct. 5, 2021 (GB) 2114226.0

(51) Int. Cl. C11D 17/00; C11D 3/50

(52) U.S. Cl. CPC C11D 17/0039; C11D 3/50; C11D 2111/12

(57) ABSTRACT

The present invention provides microcapsules comprising a hydrophobic core within a polymeric shell, wherein: a) the polymeric shell is formed from shell components comprising: i) 50% to 85% by weight, on the basis of the total weight of shell components in the microcapsules, of a polyisocyanate, wherein the polyisocyanate is an oligomer of xylylene diisocyanate and wherein the polyisocyanate comprises at least 4 isocyanate groups; ii) an alkyl silicate; iii) a polyethyleneimine; and iv) optionally, other shell components; and b) the core comprises a fragrance material. The invention further provides home or personal care formulations comprising the microcapsules and a process of producing the microcapsules.

### FIELD OF THE INVENTION

The present invention relates to microcapsules comprising a fragrance material, a home care formulation or personal care formulation comprising the microcapsules and a process of forming the microcapsules.

### BACKGROUND

Microencapsulation systems are known for encapsulating active substances such as fragrance materials. The encapsulation process results in a microcapsule comprising a core of active substance surrounded by a polymer shell. Often the active substance is hydrophobic which allows the shell to be polymerized around particles (for example, droplets) of the hydrophobic substance which are dispersed and/or emulsified in aqueous medium and/or solvent.

Various methods for making core-and-shell microcapsules have been proposed in the literature. For instance, it is known to encapsulate a hydrophobic core substance by dispersing the core substance into an aqueous medium containing a melamine formaldehyde (MF) pre-condensate and then reducing the pH to produce a microcapsule comprising an aminoplast resin shell wall surrounding the core substance. EP2794839B discloses aminoplast microcapsules which are stabilized by a polyisocyanate.

Encapsulation of active substances is also known using a plurality of polyisocyanates. Examples of multiple polyisocyanate encapsulation include EP2399667B which discloses a process for producing microcapsules in which it is essential to the process that at least two structurally different isocyanates (A) and (B) are used. EP2399667B states at that the active substance it encapsulates excludes fragrance materials.

A need exists to provide improved microcapsules or to address one or more disadvantages of the prior art.

### SUMMARY OF THE INVENTION

The present invention is based in part on the recognition by the inventors that microcapsules with improved fragrance release performance may be obtained by the use of shell components comprising i) a polyisocyanate, wherein the polyisocyanate is an oligomer of xylylene diisocyanate and wherein the polyisocyanate comprises at least 4 (free) isocyanate groups, ii) an alkyl silicate and iii) a polyethyleneimine. Without being bound by theory, the alkyl silicate may help to improve the stability of the capsules and the combination of these shell components may improve fragrance release performance when compared with other microcapsules. Due to the presence of the polyisocyanate and the polyethyleneimine, the polymeric shell may be classed as a polyurea.

Viewed from a first aspect, the present invention provides microcapsules comprising a hydrophobic core within a polymeric shell, wherein:
a) the polymeric shell is formed from shell components comprising, preferably consisting of:
   i) 50% to 85% by weight, on the basis of the total weight of shell components in the microcapsules, of a polyisocyanate, wherein the polyisocyanate is an oligomer of xylylene diisocyanate and wherein the polyisocyanate comprises at least 4 isocyanate groups;
   ii) an alkyl silicate;
   iii) a polyethyleneimine; and
   iv) optionally, other shell components; and
b) the core comprises a fragrance material.

Viewed from a second aspect, the invention provides a slurry comprising microcapsules of the first aspect, water and at least one surfactant.

Viewed from a third aspect, the invention provides a home care formulation or a personal care formulation comprising a slurry of the second aspect or microcapsules of the first aspect.

Viewed from a fourth aspect, the invention provides a process of forming microcapsules of the first aspect, wherein the process comprises the steps of:
a) forming a polymerization system comprising an aqueous phase and a dispersed oil phase, wherein the oil phase comprises said fragrance material, said polyisocyanate shell component and said alkyl silicate shell component;
b) reacting the shell components by adding said polyethyleneimine shell component to the aqueous phase to form microcapsules comprising a core of the oil phase within the polymeric shell;
c) optionally, adding a deposition additive to the surface of the microcapsules; and
d) optionally, neutralizing the microcapsules using a metal hydroxide or organic acid.

Viewed from a fifth aspect, the invention provides microcapsules obtainable by a process according to the fourth aspect.

Any aspect of the invention may include any of the features described herein regarding that aspect of the invention or any other aspects of the invention.

### DETAILED DESCRIPTION OF THE INVENTION

It will be understood that any upper or lower quantity or range limit used herein may be independently combined.

It will be understood that, when describing the number of carbon atoms in a substituent group (e.g. ‘C1 to C6’), the number refers to the total number of carbon atoms present in the substituent group, including any present in any branched groups. Additionally, when describing the number of carbon atoms in, for example fatty acids, this refers to the total number of carbon atoms including the one at the carboxylic acid, and any present in any branch groups.

Many of the chemicals which may be used to produce the present invention are obtained from natural sources. Such chemicals typically include a mixture of chemical species due to their natural origin. Due to the presence of such mixtures, various parameters defined herein can be an average value and may be non-integral.

The term ‘personal care formulation’ when used herein means a consumer product intended to be applied to the human body or any part thereof for cleansing, beautifying, or improving appearance. Personal care formulations include but are not limited to cosmetics; deodorants; bar soaps; liquid soaps; facial and body washes; facial and body cleansers; hair shampoos; hair conditioners; toothpastes; shaving creams or gels; and foot care products. A personal care formulation does not include any product for which a prescription is required.

The term ‘home care formulation’ when used herein means a consumer product for use by household and/or institutional consumers for cleaning, caring, or conditioning of the home. Home care formulations include but are not limited to detergents including laundry detergents and dishwashing detergents; conditioners including fabric conditioners; cleaning formulations including hard surface cleaners; polishes and floor finishes.

The term “polymerization system” when used herein means the aqueous phase, oil phase, shell components and all other ingredients used in producing the microcapsules.

### Microcapsules

Microcapsules according to the invention comprise a hydrophobic core within a polymeric shell, wherein:
a) the polymeric shell is formed from shell components comprising:
   i) 50% to 85% by weight, on the basis of the total weight of shell components in the microcapsules, of a polyisocyanate, wherein the polyisocyanate is an oligomer of xylylene diisocyanate and wherein the polyisocyanate comprises at least 4 isocyanate groups;
   ii) an alkyl silicate;
   iii) a polyethyleneimine; and
   iv) optionally, other shell components; and
b) the core comprises a fragrance material.

Preferably the polymeric shell is formed from shell components consisting of:
i) 50% to 85% by weight, on the basis of the total weight of shell components in the microcapsules, of a polyisocyanate, wherein the polyisocyanate is an oligomer of xylylene diisocyanate and wherein the polyisocyanate comprises at least 4 isocyanate groups;
ii) an alkyl silicate;
iii) a polyethyleneimine; and
iv) optionally, other shell components.

The microcapsules may be produced in a polymerization system. A slurry may comprise the produced microcapsules, water and at least one surfactant. Preferably the microcapsules do not comprise an aminoplast resin. As used herein, an aminoplast resin is a urea-formaldehyde (UF) or a melamine-formaldehyde (MF) resin. Both types of aminoplast resin may be undesirable for environmental reasons.

Particle size parameters of the microcapsules (e.g. D10, D50 or D90 volume mean diameter) may be measured by laser diffraction particle size analysis. The measurement may be made using a Malvern Mastersizer 3000 E with the measurement cell Hydro EV.

The microcapsules may have a D10 volume mean diameter (i.e. the point below which 10% of the microcapsules are contained, measured on a volume basis as described herein) of at least 0.5 μm, preferably at least 1 μm, more preferably at least 1.5 μm. The microcapsules may have a D10 volume mean diameter of at most 30 μm, preferably at most 20 μm, more preferably at most 10 μm.

The microcapsules may have a D50 volume mean diameter, measured as described herein, of at least 2 μm, preferably at least 4 μm, more preferably at least 5 μm, yet more preferably at least 5.5 μm. The microcapsules may have a D50 volume mean diameter of at most 50 μm, preferably at most 40 μm, more preferably at most 30 μm, yet more preferably at most 20 μm.

The microcapsules may have a D90 volume mean diameter, measured as described herein, of at least 9 μm, preferably at least 12 μm, particularly at least 15 μm. The microcapsules may have a D90 volume mean diameter of at most 80 μm, preferably at most 60 μm, particularly at most 40 μm.

Shell Component i)—Polyisocyanate

The microcapsules comprise a polymeric shell. A component of the polymeric shell is a polyisocyanate. The polyisocyanate is an oligomer of xylylene diisocyanate (XDI). The polyisocyanate comprises at least 4 isocyanate groups. The isocyanate groups may be free isocyanate groups i.e. isocyanate groups which are unreacted within the polyisocyanate. The polyisocyanate may comprise at most 8 isocyanate groups, preferably at most 6 isocyanate groups. The isocyanate groups may be free isocyanate groups. The polyisocyanate may be available as Takenate D131 (N) from Mitsui Chemicals. Preferably the polyisocyanate is not a polyol adduct of XDI. The polyisocyanate may not comprise a trimethylol propane (TMP) adduct of XDI (such as Takenate D110 (or D110-N) from Mitsui Chemicals). The polyisocyanate is not Takenate D110 (N). Takenate D110 has 3 free isocyanate groups which is less than the at least 4 isocyanate groups required for the polyisocyanate of the invention. The polyisocyanate of the invention may advantageously have an improved pot life, improved heat resistance or less tendency for yellowing than alternative polyisocyanates such as a TMP adduct of XDI (such as Takenate D110).

The polymeric shell comprises 50% to 85% by weight of the polyisocyanate, on the basis of the total weight of shell components in the microcapsules. The polymeric shell may comprise at least 55%, preferably at least 60% by weight of the polyisocyanate, on the basis of the total weight of shell components in the microcapsules. The polymeric shell may comprise at most 80%, preferably at most 75% by weight of the polyisocyanate, on the basis of the total weight of shell components in the microcapsules.

The amount of polyisocyanate in the microcapsules may also be specified by reference to the amount of polyisocyanate included in the polymerization system, for example see Table 1 in Example 1 below. The polymerization system may comprise at least 0.5 wt % of polyisocyanate on the basis of the total weight of the polymerization system, preferably at least 1 wt %, more preferably at least 1.5 wt %, particularly at least 2 wt %. The polymerization system may comprise at most 5 wt % of polyisocyanate on the basis of the total weight of the polymerization system, preferably at most 4 wt %, more preferably at most 3 wt %, particularly at most 2.5 wt %.

The weight ratio of polyisocyanate to alkyl silicate in the polymeric shell may be at least 1:1, preferably at least 1.5:1, more preferably at least 2:1. The weight ratio of polyisocyanate to alkyl silicate in the polymeric shell may be at most 8:1, preferably at most 6:1, more preferably at most 4:1. The weight ratio of polyisocyanate to alkyl silicate in the microcapsules may be at least 1:1, preferably at least 1.5:1, more preferably at least 2:1. The weight ratio of polyisocyanate to alkyl silicate in the microcapsules may be at most 8:1, preferably at most 6:1, more preferably at most 4:1. The weight ratio of polyisocyanate to alkyl silicate in the polymerization system may be at least 1:1, preferably at least 1.5:1, more preferably at least 2:1. The weight ratio of polyisocyanate to alkyl silicate in the polymerization system may be at most 8:1, preferably at most 6:1, more preferably at most 4:1.

A weight ratio of polyisocyanate to alkyl silicate of at least 1:1 advantageously allows easier microcapsule rupture and therefore improves fragrance release performance after rubbing as can be seen by comparing Microcapsules 1 and Microcapsules B in the Examples below.

Shell Component ii)—Alkyl Silicate

Another component of the polymeric shell is an alkyl silicate. Preferably the alkyl silicate is polymeric. Preferably the alkyl silicate is ethyl silicate, more preferably ethyl silicate polymer. Preferably the alkyl silicate is not tetraethyl orthosilicate monomer. The alkyl silicate may be selected from Wacker TES 40 WN and Dynasylan 40. The alkyl silicate may react during the formation of the polymeric shell to provide a polymeric silica (SiO₂) structure in the shell. The fast speed of this reaction may provide an advantage during production of the microcapsules. The microcapsules may comprise a polymeric silica structure. The polymeric shell may comprise a polymeric silica structure. The combination of polyisocyanate and alkyl silicate in the shell components may advantageously provide beneficial characteristics to the microcapsules. The alkyl silicate may increase the heat resistance of the microcapsules. The alkyl silicate may provide stability to the microcapsule slurry formed during production of the microcapsules. Without being bound by theory the alkyl silicate may provide stability to the microcapsules when they are present in a formulation which comprises the microcapsules and one or more surfactants. The surfactant(s) may be selected from anionic, cationic, non-ionic and zwitterionic sufactants, preferably anionic and cationic surfactants. The alkyl silicate may improve the tolerance of the microcapsules to such surfactants. Many home care and personal care formulations include such surfactants, for example fabric detergents and fabric softeners.

Preferably the polymeric shell comprises at least 10% by weight of the alkyl silicate, on the basis of the total weight of shell components in the microcapsules, more preferably at least 15%, yet more preferably at least 20%, particularly at least 25%. Preferably the polymeric shell comprises at most 40% by weight of the alkyl silicate, on the basis of the total weight of shell components in the microcapsules, more preferably at most 35%.

Preferably the polymeric shell comprises 10% to 40% of the alkyl silicate, by weight, on the basis of the total weight of shell components in the microcapsules.

The amount of alkyl silicate in the microcapsules may also be specified by reference to the amount of alkyl silicate included in the polymerization system, for example see Table 1 in Example 1 below. The polymerization system may comprise at least 0.2 wt % of alkyl silicate on the basis of the total weight of the polymerization system, preferably at least 0.5 wt %, more preferably at least 0.8 wt %. The polymerization system may comprise at most 4 wt % of alkyl silicate on the basis of the total weight of the polymerization system, preferably at most 3 wt %, more preferably at most 2 wt %.

Shell Component iii)—Polyethyleneimine

Another component of the polymeric shell is a polyethyleneimine. Any molecular weight and any degree of crosslinking or branching of this polymer can be used in the present invention. Preferably the polyethyleneimine has a branched structure. Preferably the polyethyleneimine has a weight average molecular weight in the range from 500 to 5,000 g/mol. Preferably the polyethyleneimine is cationic. A cationic polyethyleneimine may advantageously facilitate retention of the microcapsules on fibrous surfaces e.g. fabrics or hair.

Suitable polyethyleneimines are available from BASF (Ludwigshafen, Germany) under the LUPASOL grades (e.g., Lupasol FG, Lupasol G20 waterfree, Lupasol PR 8515, Lupasol WF, Lupasol FC, Lupasol G20, Lupasol G35, Lupasol G100, Lupasol G500, Lupasol HF, Lupasol PS, Lupasol HEO 1, Lupasol PN50, Lupasol PN60, Lupasol PO100 and Lupasol SK). Preferably the polyethyleneimine is LUPASOL PR 8515.

Preferably the polymeric shell comprises at least 0.5% by weight of the polyethyleneimine, on the basis of the total weight of shell components in the microcapsules, more preferably at least 1%, yet more preferably at least 1.5%, particularly at least 2%. Preferably the polymeric shell comprises at most 20% by weight of the polyethyleneimine, on the basis of the total weight of shell components in the microcapsules, more preferably at most 15%, yet more preferably at most 10%. Preferably the polymeric shell comprises 2% to 10% of the polyethyleneimine, by weight, on the basis of the total weight of shell components in the microcapsules.

The amount of polyethyleneimine in the microcapsules may also be specified by reference to the amount of polyethyleneimine included in the polymerization system, for example see Table 1 in Example 1 below. The polymerization system may comprise at least 0.05 wt % of polyethyleneimine on the basis of the total weight of the polymerization system, preferably at least 0.1 wt %, more preferably at least 0.15 wt %. The polymerization system may comprise at most 3 wt % of polyethyleneimine on the basis of the total weight of the polymerization system, preferably at most 2 wt %, more preferably at most 1 wt %, particularly at most 0.5 wt %.

### Optional Other Shell Components iv)

In addition to the polyisocyanate, alkyl silicate and polyethyleneimine, the polymeric shell may optionally comprise one or more other shell components. Such components are known to a person skilled in the art and may include additional polymers that can be added to the shell during the formation of the microcapsules. Preferably the optional other shell components comprise at least one polymer. Such polymers include, for example polyamines and polyquaterniums. In certain embodiments, the at least one polymer may be selected from amphoteric and cationic polymers having a weight average molecular weight in the range of from 1,000 to 1,000,000 g/mol, preferably from 10,000 to 500,000 g/mol.

### Microcapsule Core

The microcapsule core comprises a fragrance material. The core is hydrophobic overall. Preferably the fragrance material is hydrophobic.

The core may comprise at least 40% of fragrance material by weight on the basis of the total weight of the core, preferably at least 60%, particularly at least 70%, desirably at least 80% and especially at least 90%. The core may comprise 100% of fragrance material by weight on the basis of the total weight of the core. The core may consist essentially of the fragrance material.

Preferably, the fragrance material is a mixture of at least one fragrance compound and at least one solvent. The nature and type of the fragrance compounds present in the microcapsules do not warrant a detailed description here, which in any case would not be exhaustive, the skilled person being able to select them on the basis of its general knowledge and according to the intended use or application and the desired organoleptic effect. Typically, the fragrance material is a mixture of fragrance compounds. The fragrance compounds may belong to chemical classes as varied as alcohols, aldehydes, ketones, esters, ethers, acetates, nitriles, terpenoids, nitrogenous or sulphurous heterocyclic compounds and essential oils, and said materials can be of natural or synthetic origin. Many of these fragrance compounds are listed in reference texts such as the book by S. Arctander, Perfume and Flavor Chemicals, 1969, Montclair, New Jersey, USA, or its more recent versions, the relevant parts of which are incorporated herein by reference.

Preferably the fragrance material comprises at least one fragrance compound selected from:
i) hydrocarbons;
ii) aliphatic alcohols;
iii) aliphatic ketones and oximes thereof;
iv) aliphatic carboxylic acids and esters thereof;
v) acyclic terpene alcohols;
vi) acyclic terpene aldehydes and ketones;
vii) cyclic terpene alcohols;
viii) cyclic terpene aldehydes and ketones;
ix) cyclic alcohols;
x) cycloaliphatic alcohols;
xi) cyclic and cycloaliphatic ethers;
xii) (ethoxymethoxy)cyclododecane;
xiii) cyclic ketones;
xv) esters of cyclic alcohols;
xvi) esters of cycloaliphatic carboxylic acids;
xvii) aromatic and aliphatic alcohols;
xviii) esters of aliphatic alcohols and aliphatic carboxylic acids;
xix) aromatic and aliphatic aldehydes;
xxi) aromatic and aliphatic carboxylic acids and esters thereof;
xxii) nitrogen-containing aromatic compounds;
xxiii) phenols, phenyl ethers and phenyl esters;
xxiv) heterocyclic compounds;
xxv) lactones; and
xxvi) essential oils.

Preferably the fragrance material comprises at least one solvent. The solvent may assist the encapsulation of the fragrance material by assisting the fragrance material to remain in the core phase during polymerization of the shell. The solvent may comprise at least one ester.

The solvent may be a hydrophobic material that is miscible with the fragrance compounds. The solvent may provide at least one of the following benefits: i) increase the compatibility of fragrance compounds in the fragrance material, ii) increase the overall hydrophobicity of the core, iii) influence the vapor pressure of the core, and iv) provide rheological structure to the core. Suitable solvents are those having reasonable affinity for the fragrance compounds. The affinity may be determined by using a group contribution method to predict a partition co-efficient which can be expressed by a ClogP value. Preferably the solvent has a ClogP greater than 2.5, preferably greater than 3.5 and more preferably greater than 5.5. It should be noted that selecting a solvent and an overall fragrance material with high affinity for each other will result in an improvement in the stability of the core.

It is preferred that the fragrance compounds in the fragrance material have a ClogP of 0.5 to 15. Preferably the fragrance material has a weight-averaged ClogP of at least 2. The use of fragrance compounds to make a fragrance material with a weight-averaged ClogP of at least 2 is likely to be suitable for encapsulation. The fragrance compounds are generally water-insoluble, and may be delivered through the microcapsules of this invention onto consumer products in different stages such as damp and dry fabric.

Without encapsulation, the free fragrance compounds may evaporate or dissolved in water during use, e.g., during a wash cycle. Higher ClogP fragrance compounds are generally well delivered from a regular (non-encapsulated) fragrance in a consumer product, but are also suitable for encapsulation for overall fragrance character purposes, longer lasting fragrance delivery, or overcoming incompatibility with the consumer product. For example. fragrance compounds that would otherwise be instable, cause thickening or discoloration of the product or otherwise negatively affect desired consumer product properties can be encapsulated to overcome such disadvantages.

The amount of fragrance material in the microcapsules may be specified by reference to the amount of fragrance material included in the polymerization system, for example see Table 1 in Example 1 below. The polymerization system may comprise at least 10 wt % of fragrance materials on the basis of the total weight of the polymerization system, preferably at least 15 wt %, more preferably at least 20 wt %, particularly at least 25 wt %. The polymerization system may comprise at most 45 wt % of fragrance materials on the basis of the total weight of the polymerization system, preferably at most 40 wt %, more preferably at most 35 wt %, particularly at most 30 wt %.

### Deposition Additive

Preferably the microcapsules further comprising a deposition additive on their surface. The deposition additive may be polymeric. The deposition additive may comprise a hydrolysed protein. Preferably the deposition additive is cationic. A cationic deposition additive may assist the deposition of the microcapsules on fibrous surfaces such as textiles or hair. Preferably the deposition additive comprises a quaternary nitrogen group. Preferably the deposition additive is a polyquaternium, more preferably polyquaternium-11 (available as Luviquat PQ11 from BASF). The deposition additive may be selected from Luviquat PQ11, Lupamin 9030, Salcare SC60, SoftCAT SX 1300 X, Jaguar C17, Merquat 550. The deposition additive may be added to the polymerization system during the process of making the microcapsules, preferably after the microcapsules have been formed. Preferably the polymerization system is heated after the deposition additive is added to bind the deposition additive to the surface of the microcapsules.

The polymerization system may comprise at least 2 wt % deposition additive on the basis of the total weight of the polymerization system, preferably at least 4 wt %, more preferably at least 6 wt %, particularly at least 8 wt %. The polymerization system may comprise at most 20 wt % deposition additive on the basis of the total weight of the polymerization system, preferably at most 18 wt %, more preferably at most 16 wt %, particularly at most 14 wt %.

### Polymerization System

Preferably the microcapsules are produced in a polymerization system. The polymerization system may comprise an aqueous phase and an oil phase. The oil phase may be a dispersed and/or emulsified oil phase. The aqueous phase, oil phase, shell components and all other ingredients used in the process of forming the microcapsules will be referred to herein as the ‘polymerization system’.

One or more components of the microcapsule may be present in the oil phase. Preferably the fragrance material is present in the oil phase. Preferably the polyisocyanate is present in the oil phase. Preferably the alkyl silicate is present in the oil phase.

During formation of the microcapsules, the shell components are reacted to form the polymeric shell around the core. Reacting the shell components preferably forms microcapsules comprising a core of the oil phase within a polymeric shell. The core comprises the fragrance material.

The polymerization system may further comprise one or more emulsifiers and/or other surfactants. An emulsifier, which may have a high HLB (preferably HLB of 10 to 20, more preferably 15 to 20), may be dissolved into the aqueous phase to assist emulsification of the oil phase.

The polymerization system may further comprise at least one additive to assist the production of the microcapsules. The additive may comprise a hydrophilic polymer, for example a polymer containing pendant hydroxyl groups, for instance a polyvinyl alcohol. The additive may comprise a carboxyalkylcellulose, preferably carboxymethylcellulose, particularly sodium carboxymethylcellulose. Preferably the at least one additive comprises polyvinyl alcohol. The at least one additive may comprise sodium carboxymethylcellulose. The polyvinyl alcohol may be used in aqueous solution. The polyvinyl alcohol may be derived from polyvinyl acetate, and preferably between 75 and 99% of the vinyl acetate groups are hydrolyzed to vinyl alcohol units.

The polymerization system may comprise at least 0.1 wt % polyvinyl alcohol on the basis of the total weight of the polymerization system, preferably at least 0.2 wt %, more preferably at least 0.3 wt %. The polymerization system may comprise at most 3 wt % polyvinyl alcohol on the basis of the total weight of the polymerization system, preferably at most 2 wt %, more preferably at most 1 wt %.

The polymerization system may comprise at least 0.05 wt % carboxymethylcellulose on the basis of the total weight of the polymerization system, preferably at least 0.1 wt %. The polymerization system may comprise at most 2 wt % carboxymethylcellulose on the basis of the total weight of the polymerization system, preferably at most 1.5 wt %, more preferably at most 1 wt %.

The polymerization system may comprise at least 1 wt % urea on the basis of the total weight of the polymerization system, preferably at least 2 wt %. The polymerization system may comprise at most 8 wt % urea on the basis of the total weight of the polymerization system, preferably at most 6 wt %, more preferably at most 4 wt %.

The polymerization system may comprise at least 0.1 wt % xanthan gum on the basis of the total weight of the polymerization system, preferably at least 0.15 wt %. The polymerization system may comprise at most 2 wt % xanthan gum on the basis of the total weight of the polymerization system, preferably at most 1.5 wt %, more preferably at most 1 wt %.

The microcapsules may be produced in the form of a slurry. The slurry may comprise the microcapsules, water and at least one surfactant.

### Process of Forming the Microcapsules

A process of forming microcapsules according to the invention comprises the steps of:
a) forming a polymerization system comprising an aqueous phase and a dispersed oil phase, wherein the oil phase comprises said fragrance material, said shell component i) and said shell component ii);
b) reacting the shell components by adding said shell component iii) to the aqueous phase to form microcapsules comprising a core of the oil phase within the polymeric shell;
c) optionally, adding a deposition additive to the surface of the microcapsules; and
d) optionally, adjusting the pH of the microcapsules using a metal hydroxide or organic acid.

Preferably the polymerization system comprises a polyvinyl alcohol. The polymerization system may comprise a carboxymethylcellulose, preferably sodium carboxymethylcellulose. The polymerization system may comprise any of the features of a polymerization system described herein. Once the microcapsules have been formed by polymerization, one or more optional post-polymerization steps may be taken.

Preferably the process comprises step c) adding a deposition additive to the surface of the microcapsules. Preferably the deposition additive is a cationic polymer. The deposition additive may comprise any of the features of a deposition additive described herein.

Preferably the process comprises step d) neutralizing the microcapsules using a metal hydroxide or organic acid. Preferably the pH of the microcapsules is adjusted to between 7 and 8. Preferably the metal hydroxide is NaOH and/or the organic acid is formic acid.

If acidic shell monomers are used, the resulting microcapsules or microcapsule slurry may be acidic. Such microcapsules may be neutralized by the use of a hydroxide, preferably a metal hydroxide, more preferably an alkali metal hydroxide, particularly sodium hydroxide. Examples of suitable alkali metal hydroxides are NaOH and KOH. The microcapsules may also be neutralized by using an amine, for example ammonia, monoethanolamine, diethanolamine or triethanolamine, preferably ammonia. Alternatively, if the pH is too high, the microcapsules may be neutralized using an organic acid, preferably a weak organic acid, more preferably formic acid.

Preferably the process comprises the step of adding urea after the microcapsules have been formed. The urea may improve the curing efficiency and/or the physico-chemical and isolation properties of the microcapsules.

Preferably the process comprises the step of adding xanthan gum after the microcapsules have been formed. The xanthan gum may increase viscosity of the slurry and improve stabillity of the microcapsules, for example by preventing separation of the slurry.

### Personal Care & Home Care Formulations

According to one aspect, the invention provides a personal care formulation comprising microcapsules or a slurry according to the invention. Preferably the personal care formulation is for topical application to skin or hair.

The personal care formulation may be selected from hand soaps; bar soaps; liquid soaps; facial and body washes; personal care cleansers; shampoos; conditioners; toothpaste; shaving creams or gels; foot care products, moisturizers, sunscreens, after sun products, body butters, gel creams, high perfume containing products, perfume creams, baby care products, hair treatments, hair colourants, skin toning and skin whitening products, water-free products, anti-perspirant and deodorant products, tanning products, 2-in-1 foaming emulsions, multiple emulsions, preservative free products, mild formulations, scrub formulations e.g. containing solid beads, silicone in water formulations, pigment containing products, sprayable emulsions, cosmetics, colour cosmetics, conditioners, shower products, foaming emulsions, make-up remover, eye make-up remover, and wipes. Preferably the personal care formulation is selected from hair care products, skin care products, cosmetics, personal care cleansers, deodorants and anti-perspirants.

The personal care formulation preferably comprises microcapsules or a slurry according to the invention and at least one additional personal care ingredient. The personal care ingredient may be selected from a cleaning agent, hair conditioning agent, hair styling agent, anti-dandruff agent, hair growth promoter, perfume, sunscreen, sunblock, pigment, moisturizer, film former, hair color, make-up agent, thickening agent, emulsifier, humectant, emollient, antiseptic agent, deodorant active, dermatologically acceptable carrier, surfactant, abrasive, absorbent, fragrance, colorant, essential oil, astringent, anti-acne agent, anti-caking agent, anti-foaming agent, anti-oxidant, binder, enzyme, enzyme inhibitor, enzyme activator, coenzyme, botanical extract, ceramide, buffering agent, bulking agent, chelating agent, cosmetic biocide, external analgesic, substantivity increasing agent, opacifying agent, pH adjuster, reducing agent, sequestrant, skin bleaching and/or lightening agent, skin conditioning agent, skin soothing and/or healing agent, skin treating agent, vitamin or preservative. Preferably the personal care ingredient is selected from a cleaning agent, hair conditioning agent, skin conditioning agent, hair styling agent, antidandruff agent, hair growth promoter, perfume, sunscreen compound, pigment, moisturizer, film former, humectant, alpha-hydroxy acid, hair colour, make-up agent, thickening agent, antiseptic agent, deodorant, surfactant. The personal care formulation may comprise microcapsules according to the invention and at least one surfactant. The at least one surfactant may be selected from anionic, cationic, non-ionic and zwitterionic surfactants, preferably anionic and cationic surfactants.

According to one aspect, the invention provides a home care formulation comprising microcapsules or a slurry according to the invention. Preferably the home care formulation is for application to fabric or textile.

The home care formulation may be selected from fabric detergents (in liquid, powder, concentrated, unit dose or tablet form), fabric softeners (in liquid, powder, concentrated, unit dose or tablet form), fabric wash additives, fabric scent boosters (in liquid, gel, tablet, powder or granule form), refresher sprays, air care products, cleaning products, fabric cleaners, fabric conditioners, stain removers, hard surface cleaners, hand dishwashing detergents, machine dishwashing detergents, polishes and floor finishes. Preferably the home care formulation is selected from fabric conditioners, fabric detergents, fabric softeners, fabric wash additives, fabric scent boosters, refresher sprays, air care products and cleaning products.

The home care formulation preferably comprises microcapsules or a slurry according to the invention and at least one additional home care ingredient. The home care ingredient may be selected from surfactants, builders, chelating agents, dye transfer inhibiting agents, dispersants, enzymes, enzyme stabilizers, catalytic materials, bleach activators, hydrogen peroxide, sources of hydrogen peroxide, preformed peracids, polymeric dispersing agents, soil removal/anti-redeposition agents, brighteners, suds suppressors, dyes, fabric softeners, carriers, structurants, hydrotropes, processing aids, solvents and/or pigments and mixtures thereof, preferably the home care ingredient is selected from the group consisting of surfactants, builders, chelating agents, fabric softeners. The home care formulation may comprise microcapsules according to the invention and at least one surfactant. The at least one surfactant may be selected from anionic, cationic, non-ionic and zwitterionic surfactants, preferably anionic and cationic surfactants.

Any or all the features described herein, and/or any or all of the steps of any method or process described herein, may be used in any combination in any aspect of the invention.

### EXAMPLES

The invention is illustrated by the following non-limiting examples. It will be understood that all test procedures and physical parameters described herein have been determined at atmospheric pressure and room temperature (i.e. about 25° C.), unless otherwise stated herein, or unless otherwise stated in the referenced test methods and procedures. All parts and percentages are given by weight unless otherwise stated.

### Test Methods

In this specification, the following test methods have been used:
(i) Particle size analysis (including measurement of D10, D50, D90 volume mean diameters) of the microcapsules was performed using a Malvern Mastersizer 3000E with supplied software and the measurement cell Hydro EV. This is a laser diffraction particle size analysis equipment which uses the Mie theory and the refractive index of the sample to determine the particle size distribution. The microcapsule sample is thoroughly mixed and then diluted into water for the particle size measurements to be taken. Various particle size parameters and distributions are automatically measured.
(ii) Fragrance release performance of the microcapsules in a fabric softener and in a liquid laundry detergent was evaluated using a towel washing protocol and scoring by panellists. The fabric softener and liquid detergent were made according to standard formulations to which a controlled amount of microcapsules were added. One towel per sample is hand washed (at 40° C.) in the formulation containing the microcapsules. All towels are evaluated after drying for 24 hours. The aim of these evaluations is to determine the effectiveness of the fragrance release by the microcapsules after rubbing the dry towel. The panellists recorded their score for fragrance performance before rubbing and after rubbing. The scoring system was from 1 to 10 with a higher score indicating a higher fragrance intensity and better performance. The detailed procedures for the evaluation of the fabric softener and liquid detergent are given below:

### Fabric Softener

1) Depending on the number of panellists, 10 g of sample are prepared and used for each towel.

2) Dilute 10 grams of fabric softener into 1 litre at 40° C.

3) Stir with a spatula to dissolve product evenly through the water.

4) Put the towels in and rub the towels around 5 times against each other (washer should wear disposable gloves) and let it soak for 25 minutes.

5) Once the time has been completed, the towels are wrung out and left to dry for 24 hours before being evaluated (Hang out towel in an unperfumed atmosphere to dry).

### Liquid Laundry Detergent

The method is like the softener one with the difference that after step 4, and when the 25 minutes are over, the towels are rinsed (5 times) with 1 litre of water at 40° C. for each towel used.

### Example 1

Microcapsules according to the invention (Microcapsules 1) were synthesised using the materials listed in Table 1.

TABLE 1
Polymerization system for Microcapsules 1

| | | Amount |
|---|---|---|
| | | (wt % of total |
| | | polymerization |
| Material | Description | system) |
| Fragrance | Fragrance compounds | 29.70 |
| Material 1 | & solvents | |
| Takenate | Polyisocyanate | 2.15 |
| D131N | (XDI trimer) | |
| Wacker TES40 | Ethyl silicate polymer | 1.00 |
| Water | | 53.18 |
| Poval 18-88 | Polyvinyl alcohol | 0.34 |
| Finnfix 5 | Sodium Carboxymethylcellulose | 0.15 |
| Lupasol PR8515 | Polyethyleneimine | 0.20 |
| NaHCO₃ | Sodium bicarbonate | 0.33 |
| Luviquat PQ11 | Polyquaternium D11 | 10.00 |
| | (Deposition additive) | |
| Urea | | 2.75 |
| Keltrol RD | Xanthan gum | 0.20 |
| | TOTAL | 100.00 |

Microcapsules 1 were formed using the materials of Table 1 as follows. Pre-mix I was prepared from 4.42 g of Poval 18-88, 600.60 g of water and 1.95 g of Finnfix 5 with heating, if necessary, until completely dissolved. Pre-mix II was prepared from 386.10 g of Fragrance Material 1 (containing standard fragrance compounds and solvents available from Iberchem), 27.95 g of Takenate D131N (ex Mitsui Chemicals) and 13.00 g of Wacker TES40 (ex Wacker), added dropwise. The two pre-mixes I & II were combined and emulsified with the help of an Ultraturrax T25 at room temperature at a speed of 9000 rpm. The pH of the emulsion was then adjusted to 2.5 using aqueous hydrochloric acid solution (10% strength by weight). Then, at 35° C. and with stirring at 150 l/min, a solution of 2.60 g of Lupasol PR8515 (polyethyleneimine, ex BASF) in a sodium bicarbonate solution at 7% in water was added to the emulsion over the course of 2 hours. The reaction mixture was then subjected to the following temperature program: heating to 55° C. after those 2 hours, maintaining this temperature for 2 hours, then 3 hours at 80° C. After this time, add the 130.0 g of Luviquat PQ11 (ex BASF) and allow to mix for 15 minutes with Ultraturrax T25 at 8000 rpm. Then prepare a premix of urea and water (35.75 g of each) with heat at 60° C., add it to the mixture and when it dissolves add 2.6 g of Keltrol RD to the mixture with stirring and turn off the heating plate. The mixture was then cooled to room temperature. Finally, the pH of the produced microcapsule slurry was adjusted to 7.5 using aqueous sodium hydroxide solution or formic acid solution.

The produced microcapsule slurry will be referred to as Microcapsules 1.

### Example 2 (Comparative)

Comparative aminoplast resin (melamine-formaldehyde) microcapsules (Microcapsules A) not according to the invention were produced using the same core fragrance material as Microcapsules 1 but using the ingredients of Table 2 to form the microcapsules following methods known in the art.

TABLE 2
Polymerization system for Microcapsules A

| | | Amount |
|---|---|---|
| Material | Description | (wt %) |
| Water | | To 100 |
| Luracoll SD | methoxy-methylolated | 6.08 |
| | melamine pre-condensate | |
| | (monomer) | |
| Lupasol PA140 | Copolymer of | 9.00 |
| | acrylamidopropylsulfonate | |
| | and methacrylic | |
| | acid/acrylic acid. | |
| Fragrance Material 1 | | 37.00 |
| HCHO (formaldehyde) | | 3.00 |
| Scavenger | | |
| Acid | | 0.33 |
| Alkali pH Adjuster | | 0.77 |
| Deposition Additive | | 0.10 |

Microcapsules A were produced as follows:
1. Weigh water into process vessel
2. Start stirrer
3. Add Luracoll SD and Lupasol PA140 (available ex BASF)
4. Add Fragrance Material 1
5. Heat to 35°-60° C.
6. Start high shear mixing using ultraturax
7. Adjust pH to acid range
8. Increase the temperature to 85° C. over a period of 2 hours
9. Switch off ultraturrax when particle size is within desired range
10. Check the pH if the pH and readjust to acid range if necessary
11. Add the deposition additive
12. Cool to 30° C.
13. Add the HCHO (formaldehyde) scavenger with stirring
14. Mix for 15 min
15. Add alkali and adjust the pH to specification range.

The produced microcapsule slurry will be referred to as Microcapsules A.

### Example 3

Microcapsules 1 from Example 1 and Microcapsules A from Example 2 were compared as follows. Particle size analysis was performed as described in the Test Methods herein to obtain D10, D50 and D90 volume mean diameters of the microcapsules. The results are given in Table 3.

TABLE 3
Particle Size analysis

| Sample | D10 (μm) | D50 (μm) | D90 (μm) |
|---|---|---|---|
| Microcapsules 1 | 3.20 | 9.77 | 18.4 |
| Comparative Microcapsules A | 2.21 | 4.59 | 8.30 |

It can be seen from Table 3 that Microcapsules 1 of the invention have larger D10, D50 and D90 volume mean diameters. This is advantageous because as the size increases it is usually easier to produce capsule rupture, potentially resulting from a thinner capsule wall. As capsules become easier to rupture, the perfume release performance of the capsules increases.

Fragrance release performance in fabric softener was evaluated as described in the Test Methods herein. The results are given in Table 4.

TABLE 4
Fragrance release performance in Fabric Softener

| Fabric Softener | Before rubbing | After rubbing |
|---|---|---|
| Microcapsules 1 | 2 | 8 |
| Comparative Microcapsules A | 2 | 6 |

It can be seen that the score the panellists gave for fragrance release after rubbing was higher for Microcapsules 1 of the invention. This is particularly significant since the microcapsule dosage amounts in the tests were not adjusted according to the theoretical perfume load in each system i.e. the dosage by weight of microcapsule slurry was kept the same. However, as can be seen from Tables 1 & 2, Microcapsules A comprise more fragrance material than Microcapsules 1. Since the fragrance material is generally one of the most expensive parts of the composition, it can be seen that Microcapsules 1 provide an improved fragrance release performance after rubbing using a more cost-effective composition comprising less fragrance material.

### Example 4 (Comparative)

Comparative microcapsules (Microcapsules B) were made in a similar manner to Microcapsules 1 of Example 1 but using different amounts of the shell components, including a greater proportion of ethyl silicate polymer when compared with polyisocyanate. Microcapsules B were synthesised using the materials listed in Table 5.

TABLE 5
Polymerization system for Microcapsules B

| | | Amount |
|---|---|---|
| | | (wt % of total |
| | | polymerization |
| Material | Description | system) |
| Fragrance Material 1 | Oil phase | 29.70 |
| Takenate D131N | Polyisocyanate (XDI trimer) | 2.13 |
| Wacker TES40 | Ethyl silicate polymer | 3.00 |
| Water | | 50.95 |
| Poval 18-88 | Polyvinyl alcohol | 0.34 |
| Zemac E400 | Copolymer of ethylene | 0.50 |
| | and maleic anhydride | |
| Lupasol PR 8515 | Polyamine | 0.20 |
| NaHCO3 | Sodium bicarbonate | 0.33 |
| Luviquat PQ 11 | Polyquaternium D 11 | 10.00 |
| | (Deposition additive) | |
| Urea | | 2.75 |
| Keltrol RD | Xanthan gum | 0.10 |
| | TOTAL | 100.00 |

The manufacturing steps are similar to those of Microcapsules 1 in Example 1, except that the aqueous phase (Pre-Mix I) contains Zemac E400 instead of Finnfix 5 and some quantities differ, such as a 2% increase in Wacker TES40 to make it the largest amount of shell component. All other steps are the same.

### Example 5

Microcapsules 1 from Example 1 and Microcapsules B from Example 4 were compared for fragrance release performance in fabric softener and liquid laundry detergent as described in the Test Methods herein. The results are given in Tables 6 & 7.

TABLE 6
Fragrance release performance in Liquid Laundry Detergent

| Laundry | Before | After |
|---|---|---|
| Detergent | rubbing | rubbing |
| Microcapsules 1 | 2 | 7 |
| Microcapsules B | 1 | 5 |

TABLE 7
Fragrance release performance in Fabric Softener

| | Before | After |
|---|---|---|
| Fabric Softener | rubbing | rubbing |
| Microcapsules 1 | 1 | 8 |
| Microcapsules B | 1 | 6 |

Surprisingly, it can be seen from Tables 6 & 7 that Microcapsules 1 according to the invention have a better fragrance release performance than Microcapsules B in both liquid laundry detergent and fabric softener. This advantage can be attributed to the higher proportion by weight of polyisocyanate in the shell components of Microcapsules 1 (64%) when compared with Microcapsules B (40%).

It is to be understood that the invention is not to be limited to the details of the above embodiments, which are described by way of example only. Many variations are possible.

1. Microcapsules comprising a hydrophobic core within a polymeric shell, wherein:
a) the polymeric shell is formed from shell components comprising:
i) 50% to 85% by weight, on the basis of the total weight of shell components in the microcapsules, of a polyisocyanate, wherein the polyisocyanate is an oligomer of xylylene diisocyanate and wherein the polyisocyanate comprises at least 4 isocyanate groups;
ii) an alkyl silicate;
iii) a polyethyleneimine; and
iv) optionally, other shell components; and
b) the core comprises a fragrance material.

2. Microcapsules according to claim 1 wherein the polyisocyanate does not comprise a trimethylol propane adduct of xylylene diisocyanate.

3. Microcapsules according to claim 1 wherein the polymeric shell comprises 10% to 40% of the alkyl silicate, by weight, on the basis of the total weight of shell components in the microcapsules.

4. Microcapsules according to claim 1 wherein the alkyl silicate is an ethyl silicate.

5. Microcapsules according to claim 1 wherein the polymeric shell comprises 2% to 10% of the polyethyleneimine, by weight, on the basis of the total weight of shell components in the microcapsules.

6. Microcapsules according to claim 1 wherein the polyethyleneimine has a weight average molecular weight in the range from 500 to 5,000 g/mol. Patent

7. Microcapsules according to claim 1 wherein the polyethyleneimine is cationic and has a branched structure.

8. Microcapsules according to claim 1 wherein the microcapsules do not comprise an aminoplast resin.

9. Microcapsules according to claim 1 further comprising a polymeric deposition additive on the surface of the microcapsules.

10. A slurry comprising microcapsules according to claim 1, water and at least one surfactant.

11. A home care formulation comprising microcapsules according to claim 1 and at least one additional home care ingredient.

12. A home care formulation comprising microcapsules according to claim 1 wherein the home care formulation is selected from fabric conditioners, fabric detergents, fabric softeners, fabric wash additives, fabric scent boosters, refresher sprays, air care products and cleaning products.

13. A personal care formulation comprising microcapsules according to claim 1 at least one additional personal care ingredient.

14. A personal care formulation comprising microcapsules according to claim 1 wherein the personal care formulation is selected from hair care products, skin care products, cosmetics, personal care cleansers, deodorants and anti-perspirants.

15. A process of producing microcapsules according to claim 1, wherein the process comprises the steps of:
a) forming a polymerization system comprising an aqueous phase and a dispersed oil phase, wherein the oil phase comprises said fragrance material, said polyisocyanate shell component and said alkyl silicate shell component;
b) reacting the shell components by adding said polyethyleneimine shell component to the aqueous phase to form microcapsules comprising a core of the oil phase within the polymeric shell;
c) optionally, adding a deposition additive to the surface of the microcapsules; and
d) optionally, neutralizing the microcapsules using a metal hydroxide or organic acid.

16. The process according to claim 15 wherein the polymerization system further comprises a polyvinyl alcohol.

17. The process according to claim 15 wherein the polymerization system further comprises a carboxymethylcellulose.

18. The process according to claim 15 comprising step c) adding a deposition additive to the surface of the microcapsules, wherein the deposition additive is a cationic polymer.

19. The process according to claim 15 comprising step d) neutralizing the microcapsules, wherein the metal hydroxide is NaOH and/or the organic acid is formic acid.

20. The process according to claim 15 which comprises the step of adding urea to the polymerization system after the microcapsules have been formed.

21. The process according to claim 15 which comprises the step of adding xanthan gum to the polymerization system after the microcapsules have been formed

22. Microcapsules obtained by the process according to claim 15.
